(19) United States
(12) Patent Application Publication
(10) Pub. No.: US 2008/0015394 A1
(43) Pub. Date: Jan. 17, 2008

(54) Process for higher purity decabromodiphenyl oxide

(76) Inventors: Steven Bakeis, West Lafayette, IN (US); David W. Bartley, West Lafayette, IN (US); Stephen B. Falloon, Lafayette, IN (US); Timothy T. Lawlor, Lafayette, IN (US); David C. Sanders, West Lafayette, IN (US); James D. Siebecker, West Lafayette, IN (US); Larry D. Timberlake, West Lafayette, IN (US)

Correspondence Address: CHEMTURA CORPORATION, 199 Benson Road, Middlebury, CT, 06749, US

(21) Appl. No.: 11/827,172

(22) Filed: Jul. 10, 2007

Related U.S. Application Data
(60) Provisional application No. 60/830,916, filed on Jul. 14, 2006.

(51) Int. Cl. C07C 41/00

(52) U.S. Cl. 568/557

(57) ABSTRACT

A process for substantially perbrominating diphenyl ether comprising the steps of:
(A) adding the diphenyl ether to a mixture of:
   (i) a greater than 400 percent excess of the stoichiometric amount of bromine; and
   (ii) a catalytically effective amount of a Lewis acid catalyst;
(B) heating said mixture to an elevated temperature during the addition; and
(C) continuing the reaction at an elevated temperature after addition of the aromatic compound has been completed.

I claim the benefit under Title 35, United States Code, 119 to U.S. Provisional Application No. 60/830,916, filed Jul. 14, 2006 entitled PROCESS FOR HIGHER PURITY DECABROMODIPHENYL OXIDE.

### BACKGROUND OF THE INVENTION

1. Field of the Invention

The present invention relates to a process for producing decabromodiphenyl oxide (deca). More particularly, the present invention relates to a process for producing a deca product containing more than 99% of the decabromo component and less than 1% of nonabromo isomers.

2. Description of Related Art

Decabromodiphenyl oxide (deca) is a commercially available material widely used to flame retard plastic resins. Owing to its high efficiency and relatively low cost, deca is often the material of choice for protecting resins that are highly flammable and difficult to flame retard. Accordingly, deca has been known in the literature for some time and there are a variety of published processes for producing it in commercial scale quantities. Such processes describe bromination of the aromatic substrate in the presence of various kinds of reaction media and solvents. Drawbacks and advantages of these processes have been previously summarized in U.S. Pat. No. 4,287,373, which describes a particularly advantageous process for preparing deca and related compounds. This process utilizes bromine as the sole reaction medium. The object of the invention was to prepare compounds such as deca in high yield and purity. Purity is described as “substantially free from lower brominated products.” In this and other known processes, the actual assay was reported in the range of 96-98% decabromo product with the major impurity in the balance of the material being the nonabromo isomers.

This level of purity has been acceptable in the commercial product for many years and has been shown through extensive scientific study to have no adverse toxicological or environmental effects. Despite the scientific evidence, however, many industrial users remain concerned that there may be future legal regulations concerning the use of deca having the current levels of nonabromo component. Therefore, there exists a market need for a higher purity deca with a lower concentration of the nonabromo component.

U.S. Pat. No. 4,778,933 discloses a process for making decabromodiphenyl oxide that comprises: (a) initiating a feed of molten diphenyl oxide to a substantially anhydrous mixture of methylene dibromide solvent, elemental bromine (Br₂) and an aluminum trihalide catalyst at a temperature between about 10 [symbol] and about 30 [symbol] C; (b) heating the reaction mixture to about 50 [symbol] −60 [symbol] C, and while maintaining the temperature at about 50 [symbol] −60 [symbol] C, continuing the feed of molten diphenyl oxide until the total amount fed is equivalent to (i) about 0.064 to about 0.077 mole per mole of elemental bromine employed in the reaction, and (ii) about 0.2 to about 10 parts by weight per part by weight of methylene dibromide employed in the reaction; (c) steam distilling the methylene dibromide solvent and the residual bromine from the reaction mixture; (d) recovering decabromodiphenyl oxide from the distillate; and (e) drying the methylene dibromide and bromine distillate to render them suitable for reuse in the process.

It is known in the art to modify process conditions, such as elevated reaction temperatures, modest excesses of bromine, and relatively short post-hold reaction times, to maximize yield and purity. However, the limits on these conditions were rather narrow. The examples and data provided do not indicate any advantages to extending the ranges to incorporate gross stoichiometric excesses of bromine and catalysts relative to the aromatic substrate or long post-reaction hold times. Indeed, there are data that teach that there is no advantage by way of increased assay in exceeding the limits of these process parameters.

The disclosures of the foregoing are incorporated herein by reference in their entirety.

### SUMMARY OF THE INVENTION

The present invention relates to a process for producing a deca product containing more than 99% decabromo component and less than 1% nonabromo isomers. Using this process, assays of 99.99% decabromo component are possible without any additional purification, such as recrystallization, digestion, milling, grinding, sublimation or roasting.

This present invention offers advantages over other known processes that may report high assay deca. One key advantage is that the present invention provides the highest reported assay, as discussed above, but another important advantage is that this high assay can be achieved using only bromine as the reaction medium. Unlike other processes that are taught to produce high assays, the present process requires no exotic reaction media, such as oleum, and no need for the addition of organic solvents, thus eliminating the need for and expense of recovery of non-reactive materials or by-products.

In accordance with the present invention, assays as high as 99.99% have been achieved by various combinations of large molar excesses of bromine, significantly higher catalyst charges, extended post-reaction hold times, elevated reaction temperatures, and combinations of the foregoing. In addition, the effects of these conditions can be obtained by techniques such as running the reaction under superatmospheric pressure, or other simple procedural and/or equipment modifications.

More particularly, the present invention is directed to a process for substantially perbrominating diphenyl ether comprising the steps of:

(A) adding the diphenyl ether to a mixture of:
(i) a greater than 400 percent excess of the stoichiometric amount of bromine; and
(ii) a catalytically effective amount of a Lewis acid catalyst;

(B) heating said mixture to an elevated temperature during the addition; and

(C) continuing the reaction at an elevated temperature after addition of the aromatic compound has been completed.

### DESCRIPTION OF THE PREFERRED EMBODIMENTS

In accordance with the present invention, assays of more than 99% deca and less than 1% nonabromo isomers can be obtained by the use of a stoichiometric excess of bromine that is greater than 400% as the reactant and reaction medium. Desirable molar excesses for such assays are preferably in the range of from greater than 400 to about 2000%, more preferably in the range of from greater than 400 to about 1000%, and most preferably in the range of from about greater than 400 to about 600%. When bromine is used at these levels, the assay of deca nearly approaches 100%, with very short reaction and post-reaction hold times. Assays of 99.99% are possible with as little as one hour diphenyl oxide addition times and a post-reaction hold time at elevated temperature of one hour. Additional run time can further increase the assay.

In order to achieve these assays in the given reaction times, consideration must be given to catalyst choice and level. Typically, Lewis acid catalysts in the form of metals and metal-containing species have been used to promote this reaction. Examples of such catalysts include iron, iron halides, and compounds that will make iron bromide under conditions of the reaction. Additionally, other Lewis acid type metals, such as antimony, may also work. As reported in U.S. Pat. No. 4,287,373, aluminum, aluminum halides and compounds that form aluminum bromide under conditions of the reaction are generally considered the catalysts of choice for perbromination of diphenyl oxide. The levels of the catalyst have been found to be important in achieving high assays with short reaction times. For purposes of the present invention, levels in the range of about 0.1 to about 45 weight % (preferably about 4 to about 26 weight %, more preferably about 8 to about 26 weight %, most preferably from greater than 15 to about 26 weight %) metal equivalent weight of Lewis acid based on the amount of diphenyl oxide in the reaction are desirable. Lower levels can be used, of course, but catalytic activity will be lowered such that either reaction times will become prohibitively long or the high assays may not be achievable.

Further, the moisture content of the bromine is an important factor when establishing the catalyst level. As is known in the art, the presence of water in the bromine will inactivate at least a portion of catalyst and, thus, higher levels of catalyst are required to compensate for the loss.

As an alternative to having a significant excess of bromine, longer post reaction hold times can be employed to increase the assay. Using previously reported reaction stoichiometries of bromine and diphenyl oxide with standard reaction conditions, assays of 99.6% were achieved by lengthening the hold time. Again, catalyst choice and usage level appear to be important considerations, and desirable levels to achieve the required assay are in the range of about 0.1 to about 45% by weight, based on metal equivalent weight of Lewis acid relative to the diphenyl oxide charge.

As previously discussed, lower levels of catalyst may reduce the activity of the catalyst such that a very long hold time is required to achieve high assay (>99%) material. For example, hold times of up to 100 hours were not reported in the patent literature to increase the assay with low level charges of aluminum catalyst. In U.S. Pat. No. 4,287,373, it was noted that catalyst charges in the range of 0.1-10% by weight were satisfactory, but examples and data were reported using the very low end of this range. There was clearly no recognition that there were benefits to be gained, such as higher assay, by using higher catalyst charges.

The bromination reaction of the diphenyl ether using excess bromine as the reaction medium can be initiated at ambient or higher temperatures. After addition of the diphenyl ether has been completed, the temperature is maintained, or increased further, preferably at or near reflux levels, during the later stages of the bromination. In the case of the perbromination of diphenyl ether, reflux occurs at about 59°-60° C.

A possible explanation for the higher assay observed with this invention could be increased catalytic activity and/or solubility of the product and intermediate brominated species with the prescribed conditions. The brominated species generated during the reaction are known to be somewhat soluble in the bromine reaction medium. By using the high excess of bromine, more material is soluble and available for reaction. Coupling the increased soluble quantity of material with higher catalytic activity associated with increasing aluminum levels could lead to a higher assay. Likewise, with extended post-reaction hold times, the higher catalytic activity drives the reaction further to the desired deca product and results in a higher assay when compared to lower levels of catalyst and the same (or longer) hold times.

Toward that end, any procedural or mechanical change that accomplishes increasing solubility and catalytic activity is an aspect of this invention. A non-binding and non-limiting example would be using current state of the art reaction conditions under superatmospheric pressure. The increase in pressure during the post-reaction hold time would permit higher temperatures. In general, the solubilities of the brominated aromatic species increase with temperature. Therefore, higher pressure equates to higher solubility and reactivity and in accordance with the above hypothesis, higher assay of deca product. An advantage to using superatmospheric pressure during the hold time would be realization of the higher assays obtained with the extended hold and/or high bromine excesses, but with normal hold times and bromine excesses.

Another potential consideration associated with the solubility of the lower brominated species is the effect of dispersion rate of the diphenyl oxide into the reaction medium. Precipitation of partially brominated isomers is known to occur during the reaction, owing to their low solubilities in the bromine reaction medium. These isomers, such as the nonabromo, can become occluded in the precipitated particles and unavailable for further bromination using the current state of the art conditions.

Therefore, a further aspect of this invention is rapid dispersion of the diphenyl oxide substrate into the bromine/catalyst reaction medium. As employed herein, “rapid dispersion” or “rapid mixing” is intended to mean blend times of up to and including twelve seconds. While not wishing to be bound by theory, one can envision that any precipitation of underbrominated species, such as nonabromo, would result in lower particle sizes when the diphenyl oxide is rapidly dispersed. The smaller particles would be less prone to occlusion of the nonabromo isomers. Thus, the nonabromo isomers would be more readily available for further bromination and the observed result would be higher assay deca for a given set of conditions.

Another hypothesis is that rapid dispersion of diphenyl oxide would lead to a higher “apparent concentration” of bromine felt by the diphenyl oxide, giving a more rapid reaction to the deca product. In essence, rapid dispersion may be a way of simulating a high excess of bromine without actually adding the bromine into the mixture.

Rapid dispersion of the diphenyl oxide can be achieved by any number of means including, but not limited to, slow addition rates of diphenyl oxide to the bromine reaction medium, multiple addition points into the reactor, mechanical designs that increase agitation and mixing in the reaction medium, high velocity injection of the diphenyloxide into the reaction medium, use of a “diptube inside a diptube” to surround the diphenyl oxide with bromine before it hits the main reaction medium, and the like.

Without further elaboration, it is believed that one skilled in the art can, using the description herein, utilize the present invention to its fullest extent. The following examples are included to provide additional guidance to those skilled in the art in practicing the claimed invention. The examples provided are merely representative of the work that contributes to the teaching of the present application. Accordingly, these examples are not intended to limit the invention, as defined in the appended claims, in any manner.

### EXAMPLES

### Comparative Example

A 500 milliliter four-neck round bottom flask was fitted with a mechanical stirrer, a double walled reflux condenser, a thermocouple, a temperature controller, a heating mantle, and a syringe pump fitted with a Teflon needle. The flask was vented to a water trap for collection of by-product hydrogen bromide. Dry bromine (929.5 grams, 5.82 moles, 200% excess) was charged into the reaction flask, followed by 4.1 grams of aluminum chloride (0.031 mole). The reaction was stirred for 5 minutes.

Addition of 33.0 grams (0.19 mole) of diphenyl ether was initiated to the bromine-catalyst mixture at a temperature of 25 [symbol] C. The diphenyl ether addition was maintained at a constant rate by use of a syringe pump over a period of about 180 minutes. The reaction temperature was allowed to increase by way of exotherm to about 35 [symbol] C. Additional heat was applied after the diphenyl ether addition had been completed, and the reaction temperature increased to about 59 [symbol] C within about 20 minutes. After 180 minutes of post addition heating, the heat input was removed and the reaction allowed to cool to room temperature in about 90 minutes.

A two liter four-neck round bottom flask was fitted with a mechanical stirrer, a distilling head, a double walled reflux condenser, a thermocouple, a temperature controller, and a heating mantle. One liter of water and the reaction slurry were charged to the flask and the excess bromine was distilled off until a temperature of 100 [symbol] C was achieved.

Decabromodiphenyl ether was filtered from the aqueous slurry, washed with water, and dried at 100° C. in a forced air oven.

Gas chromatographic analysis of the resulting product showed decabromodiphenyl ether 96.93 area percent, nonabromodiphenyl ether isomers totaling 2.79%, octabromodiphenyl ether isomers totaling 0.25%, and heptabromodiphenyl ether isomers totaling 0.02%.

### Example 1

A two liter four-neck round bottom flask was fitted with a mechanical stirrer, a double walled reflux condenser, a thermocouple, a temperature controller, a heating mantle, and a syringe pump fitted with a Teflon needle. The flask was vented to a water trap for collection of by-product hydrogen bromide. Dry bromine (3,410 grams, 21.34 moles, 1000% excess) was charged into the reaction flask, followed by 17.9 grams of aluminum chloride (0.13 mole). The reaction was stirred for five minutes.

Addition of 33.0 grams (0.19 mole) of diphenyl ether was initiated to the bromine-catalyst mixture at a temperature of 25° C. The diphenyl ether addition was maintained at a constant rate by use of a syringe pump over a period of about 60 minutes. The reaction temperature was allowed to increase by way of exotherm to about 35° C. Additional heat was applied after the diphenyl ether addition had been completed, and the reaction temperature increased to about 59° C. within about 20 minutes. After about 60 minutes of post addition heating, the heat input was removed and the reaction allowed to cool to room temperature in about 90 minutes.

A three liter four-neck round bottom flask was fitted with a mechanical stirrer, a distilling head, a double walled reflux condenser, a thermocouple, a temperature controller, and a heating mantle. One liter of water and the reaction slurry were charged to the flask and the excess bromine was distilled off until a temperature of 100° C. was achieved.

Decabromodiphenyl ether was filtered from the aqueous slurry, washed with water, and dried at 100° C. in a forced air oven.

Gas chromatographic analysis of the resulting product showed decabromodiphenylether 99.95 area percent, nonabromodiphenyl ether isomers totaling 0.05%, with no other isomers present.

### Example 2

The procedure of Example 1 was repeated except that the amount of aluminum chloride was reduced to 6.2 grams (0.047 mole).

Gas chromatographic analysis of the resulting product showed decabromodiphenylether 99.90 area percent and nonabromodiphenyl ether 0.1%, with no other isomers present.

### Example 3

A two liter four-neck round bottom flask was fitted with a mechanical stirrer, a double-walled reflux condenser, a thermocouple, a temperature controller, a heating mantle, and a syringe pump fitted with a Teflon needle. The flask was vented to a water trap for collection of by-product hydrogen bromide. Dry bromine (3410.1 grams, 21.34 moles, 1000% excess) was charged into the reaction flask, followed by 6.5 grams of aluminum chloride (0.049 mole). The reaction was stirred for five minutes.

Addition of 33.0 grams (0.19 mole) of diphenyl ether was initiated to the bromine-catalyst mixture at a temperature of 25° C. The diphenyl ether addition was maintained at a constant rate by use of a syringe pump over a period of about 60 minutes. The reaction temperature was allowed to increase by way of exotherm to about 31° C. Additional heat was applied after the diphenyl ether addition had been completed, and the reaction temperature increased to about 59° C. within about 20 minutes. After about 24 hours of post addition heating, the heat input was removed and the reaction allowed to cool to room temperature in about 90 minutes.

A three liter four-neck round bottom flask was fitted with a mechanical stirrer, a distilling head, a double walled reflux condenser, a thermocouple, a temperature controller, and a heating mantle. One liter of water and the reaction slurry were charged to the flask and the excess bromine was distilled off until a temperature of 100° C. was achieved.

Decabromodiphenyl ether was filtered from the aqueous slurry, washed with water, and dried at 100° C. in a forced air oven.

Gas chromatographic analysis of the resulting product showed decabromodiphenylether 99.99 area percent and nonabromodiphenyl ether isomers totaling <0.01%, with no other isomers present.

### Example 4

A reaction similar to Example 3 was carried out wherein the molar % excess dry bromine was 600%, the catalyst was aluminum chloride, added at 12.7% equivalent metal weight relative to the diphenyl oxide, and the mixture was heated to reflux. Addition of diphenyl ether was initiated to the bromine-catalyst mixture at a temperature of 58° C. The diphenyl ether addition was maintained at a constant rate over a period of about 190 minutes. The reaction temperature was maintained at 56° C. during the diphenyl ether addition. After about two hours of post addition heating, the heat input was removed and the reaction allowed to cool .

The excess bromine was removed from the reaction slurry by distillation from an aqueous slurry until a temperature of 100° C. was achieved.

Decabromodiphenyl ether was filtered from the aqueous slurry, washed with water, and dried. Gas chromatographic analysis of the resulting product showed decabromodiphenylether 99.74 area percent and nonabromodiphenyl ether isomers totaling 0.26%, with no other isomers present.

In view of the many changes and modifications that can be made without departing from principles underlying the invention, reference should be made to the appended claims for an understanding of the scope of the protection to be afforded the invention.

What is claimed is:

1. A process for substantially perbrominating diphenyl ether comprising the steps of:
(A) adding the diphenyl ether to a mixture of:
(i) a greater than 400 percent excess of the stoichiometric amount of bromine; and
(ii) a catalytically effective amount of a Lewis acid catalyst;
(B) heating said mixture to an elevated temperature during the addition; and
(C) continuing the reaction at an elevated temperature after addition of the aromatic compound has been completed.

2. The process of claim 1 wherein the excess of bromine is from greater than 400% to about 2000% of the stoichiometric amount for perbromination of the diphenyl ether.

3. The process of claim 1 wherein the excess of bromine is from greater than 400% to about 1000% of the stoichiometric amount for perbromination of the diphenyl ether.

4. The process of claim 1 wherein the excess of bromine is from greater than 400% to about 600% of the stoichiometric amount for perbromination of the diphenyl ether.

5. The process of claim 1 wherein the catalyst is present in an amount of from about 0.1% to about 45% by weight, based on the metal equivalent weight relative to the amount of the diphenyl ether.

6. The process of claim 2 wherein the catalyst is present in an amount of from about 0.1% to about 45% by weight, based on the metal equivalent weight relative to the amount of the diphenyl ether.

7. The process of claim 3 wherein the catalyst is present in an amount of from about 0.1% to about 45% by weight, based on the metal equivalent weight relative to the amount of the diphenyl ether.

8. The process of claim 4 wherein the catalyst is present in an amount of from about 0.1% to about 45% by weight, based on the metal equivalent weight relative to the amount of the diphenyl ether.

9. The process of claim 1 wherein the catalyst is present in an amount of from about 4% to about 26% by weight, based on the metal equivalent weight relative to the amount of the diphenyl ether.

10. The process of claim 1 wherein the catalyst is present in an amount of from about 8% to about 26% by weight, based on the metal equivalent weight relative to the amount of the diphenyl ether.

11. The process of claim 1 wherein the Lewis acid catalyst is selected from the group consisting of iron, iron halides, iron compounds which form iron bromides under the conditions of the reaction, aluminum, aluminum halides, and aluminum compounds which form aluminum bromide under the conditions of the reaction.

12. The process of claim 1 wherein the further increased elevated temperature is reflux temperature.

13. The process of claim 1 wherein the assay of the perbrominated diphenyl ether is greater than 99% deca and less than 1% nonabromo isomers.

14. A process for substantially perbrominating diphenyl ether comprising the steps of:
(A) adding the diphenyl ether to a mixture of:
(i) an excess of from greater than 400% to about 600% of the stoichiometric amount of bromine; and
(ii) from about 8% to about 26% by weight, based on the metal equivalent weight relative to the amount of the diphenyl ether, of a Lewis acid catalyst selected from the group consisting of iron, iron halides, iron compounds which form iron bromides under the conditions of the reaction, aluminum, aluminum halides, and aluminum compounds which form aluminum bromide under the conditions of the reaction;
(B) elevating the reaction temperature to about 59° C. during the addition of the diphenyl ether; and
(C) continuing the reaction at reflux temperature after addition of the aromatic compound has been completed until the assay of the perbrominated diphenyl ether is greater than 99% deca and less than 1% nonabromo isomers.
